(19) United States
(12) Patent Application Publication
(10) Pub. No.: US 2009/0036849 A1
(43) Pub. Date: Feb. 5, 2009

(54) Absorbent Article

(75) Inventors: Ingrid Gustafson, &Aring;sa (SE); Ulrika Husmark, Molnlycke (SE); Helena Engstrom, Frolunda (SE); Asa Lennersten, Molndal (SE); Ulrika Libander, Saro (SE)

Correspondence Address: BUCHANAN, INGERSOLL & ROONEY PC, POST OFFICE BOX 1404, ALEXANDRIA, VA, 22313-1404, US

(73) Assignee: SCA HYGIENE PRODUCTS AB, Göteborg (SE)

(21) Appl. No.: 12/086,922

(22) PCT Filed: Dec. 22, 2005
(86) PCT No.: PCT/SE2005/002028

§ 371 Date: Jun. 20, 2008

(51) Int. Cl. A61L 15/36

(52) U.S. Cl. 604/360

(57) ABSTRACT

An absorbent article such as a sanitary napkin, panty liner, tampon, diaper, pant diaper, adult incontinence guard, provided with at least one capsule (8), containing a bacterial composition (11) in a lipid phase (12), which preferably contains at least one lactic acid producing bacterial strain. At least one portion of the capsule has a minimum cross dimension (a) of at least 2 mm. The capsule (8) preferably includes a core (9) containing the bacterial composition (11) in lipid phase (12) and a shell (10) preventing exposure of the core during transport and storage.

### FIELD OF INVENTION

The present disclosure refers to an absorbent article such as a sanitary napkin, panty liner, tampon, diaper, pant diaper, adult incontinence guard. More specifically it refers to such articles containing a bacterial composition. The disclosure further refers to a method for making a capsule containing a bacterial composition.

### TECHNICAL BACKGROUND

The urogenital area harbors a complex microbial ecosystem comprising more than 50 different bacterial species (Hill et al., Scand. J. Urol. Nephrol. 1984; 86 (suppl.) 23-29). The dominating species for fertile women in this area are lactic acid producing bacteria belonging to the genus Lactobacillus. These lactic acid producing members are important for retaining a healthy microbial flora in these areas, and act as probiotic bacteria with an antagonistic effect against pathogenic microbial species. Lactic acid producing bacteria inhibit growth and colonization by other microorganisms by occupying suitable niches for colonization, by forming biofilms and competing for available nutrients, thereby excluding colonization by harmful microorganisms. Also, the production of hydrogen peroxide, specific inhibiting substances, such as bacteriocines, and organic acids (including lactic acid and acetic acid) that lower the pH, inhibit colonization by other microorganisms. The microbial ecosystem of a healthy individual can be disturbed by the use of antibiotics, during hormonal changes, such as during pregnancy or use of contraceptives with estrogen, during menstruation, after menopause, in people suffering from diabetes etc. Also, microorganisms may spread from the anus to the urogenital area, thereby causing infections. This results in a disturbance of the normal microbial flora and leaves the individual susceptible to microbial infections that cause vaginitis, urinary tract infections and ordinary skin infections. Microorganisms commonly associated with these kinds of infections belong to the genera Escherichia, Enterococcus, Psedomonas, Proteus, Klebsiella, Streptococcus, Staphylococcus, Gardnerella and Candida. Women are at particular risk due to their shorter distance between the anus and the urogenital tract; specially at risk are young women, who not yet have a well developed microflora in the urogenital area and older women, who no longer have a protective flora.

One way to reduce the problems with the kinds of infections described above is to have a good personal hygiene. However, excessive use of cleaning agents not only decrease the amount of harmful microbes, but can harm the beneficial microbial flora, again render it susceptible for pathogenic species to colonize and cause infections. Alternatively, administration of lactic acid producing bacteria to the urogenital area and the skin in order to outcompete pathogenic species and facilitate reestablishment and maintenance of a beneficial microbial flora in these areas, have been found to be a successful means to treat and prevent microbial infections.

It is known through WO 84/04675 to control vulvo-vaginal infections by means of vaginal gelatin capsules containing a freeze-dried concentrate of lactic acid bacteria dispersed in a pharmaceutically active fluid carrier, for example a non-hygroscopic oil.

Microencapsulation of lactic acid bacteria used in pharmaceutical compositions for intra-vaginal administration is known through EP-B-828 499 and WO 97/29762.

It has been suggested that lactic acid producing bacteria can be delivered via absorbent products, such as diapers, sanitary napkin, incontinence guards, panty liners and tampons, as described in, for example, WO 92/13577, WO 97/02846, WO 99/17813, WO 99/45099 and WO 00/35502.

A major problem with providing products intended to be used for transfer of lactic acid producing bacteria, is that the bacteria have to retain viability during transport and storage of the products. A major problem with products comprising lactic acid producing bacteria is that the bacteria rapidly lose viability under semi-moist conditions, and it is therefore important that the products are not exposed to moisture.

With “semi-moist” conditions is meant that the water activity (a_{w}) is between about 0.2 and about 0.9. Water activity a_{w} measures the vapour pressure generated by the moisture present in a hygroscopic product.

a_{w}=p/pₛ, where:

p: partial pressure of water vapour at the surface of the product
pₛ: saturation pressure, or the partial pressure of water vapour above pure water at the product temperature

Water activity reflects the active part of moisture content or the part which, under normal circumstances, can be exchanged between the product and its environment.

Water activity is usually defined under static conditions of equilibrium. Under such conditions, the partial pressure of water vapour (p) at the surface of the product is equal to the partial pressure of water vapour in the immediate environment of the product. Any exchange of moisture between the product and its environment is driven by a difference between these two partial pressures.

One way to partly overcome this problem has been to supply products with freeze-dried lactic acid producing bacteria. However, if the bacteria in the products are not protected from moisture after manufacturing of the products, the air humidity will subsequently kill the bacteria and the shelf-life of such products will then be shortened. Another disadvantage with the direct application of dried lactic acid producing bacteria to a hygiene product, such as an absorbent product, is that transfer of the bacteria to the urogenital area will be low.

In order to overcome the problem with air humidity decreasing the shelf-life of products containing lactic acid producing bacteria it has been suggested to prepare dispersions of lactic acid producing bacteria and a hydrophobic substance, such as a fat or an oil. Research experiments have shown that storage in sterile vaseline oil results in a high level of viable lactobacilli cells after 8 months of storage (Arkadéva et al., N A. Nauchnye Doklady Vysshei Shkoly. Biologicheskie Nauki, 1983, 2:101-104). However, Stoianova et al. (Mikrobiologiia, 2000, 69:98-104), found that immersion in mineral oil was not effective to preserve viability of lactic acid producing bacteria. U.S. Pat. No. 4,518,696 describes liquid suspensions of Lactobacilli in sunflower oil for oral administration to animals. However, none of the above references are concerned with the problems associated with retaining a high viability of lactic acid producing bacteria on hygiene products to be used to administer lactic acid producing bacteria to the urogenital area of a subject.

There are additional examples of the combination lactic acid producing bacteria and an oil, although these do not describe the effect of the oil on the survival of the lactic acid producing bacteria. WO 01/13956 describes the use of pharmaceutical compositions comprising Emu oil, antimicrobial agents and/or Bacillus coagulans to be used for antimicrobial treatments. However, Bacillus coagulans is not naturally occurring in the normal human urogenital flora and is mainly used as odor inhibitor and is not adapted to improve the microbial flora in humans. Bacillus coagulans is forming spores and is therefore not sensitive for normal moisture. WO 02/28446 describes the use of an essentially hydrophobic carrier and freeze-dried lactic acid producing bacteria to prepare a distribution to be applied to an absorbent product. The hydrophobic carrier was mainly chosen to overcome problems with applying the bacteria to the absorbent product during manufacturing, but the carrier also protects the bacteria from air humidity.

In conclusion, there is still a need to develop products for delivery of lactic acid producing bacteria to the urogenital area that are convenient to use, result in efficient transfer of the bacteria to the area where they are applied and that can be stored for long time periods without loss of viability of the bacterial cells.

### OBJECT AND SUMMARY

The above defined problems are solved in the present disclosure by an absorbent article provided with at last one capsule, at least one portion of which has a minimum cross dimension of at least 2 mm, said capsule containing a bacterial composition in a lipid phase. By providing the bacteria in capsules of the above kind the bacteria are protected from moisture and have a significantly prolonged shelf-life. The bacterial composition may contain at least one lactic acid producing bacterial strain.

In one aspect said capsule has at least one portion with a minimum cross dimension of between 2-10 mm, preferably between 4 and 7 mm.

In a further aspect said article is provided with at least two capsules and preferably not more than ten capsules.

In one embodiment the capsules are located spaced apart in the article a distance of at least 5 mm. Preferably they are spaced apart in the longitudinal direction of the article.

Preferably said capsule comprises a core containing said bacterial composition in said lipid phase and a shell preventing exposure of the core during transport and storage, wherein the core and the shell are of different materials.

It is further preferred that said lipid phase is a wax and/or an oil.

In one aspect the wax is chosen from a plant wax, a mineral wax, an animal wax, a silicon wax and mixtures thereof. One example of a suitable mineral wax is a wax that contains petrolatum as a main component.

In one embodiment the shell material is a wax or a polymer. Examples of suitable shell materials are: polyamide, polyurethane, formaldehyde resin, gelatine, pectin, alginate, plant wax, mineral wax, animal wax, silicon wax and mixtures thereof.

In one aspect the core material and the shell material have different polarities, so as to prevent significant mixture of said materials in the phase boundary between the shell and the core.

In a further aspect said at least one capsule is applied on the wearer facing side of the article or below one or more layers of the article permitting penetration of the lactic acid producing bacterial strain to the wearer.

In a still further aspect said at least one capsule is applied on the wearer facing side of the article and is covered by a releasable material layer, to which it is at least partly adhered, so that upon removal of the releasable material layer the shell of the capsule will burst and expose the core material.

The disclosure also refers to a method of making a capsule containing a bacterial composition in a lipid phase, said capsule having a core and a shell, wherein the materials intended to form the core and the shell respectively are delivered through a concentric double nozzle, which produces droplets composed of an inner phase containing the core material and an outer phase containing the shell material, said droplets forming the capsules.

### BRIEF DESCRIPTION OF THE DRAWINGS

FIG. 1 is a plan view of an illustrative example of an embodiment of an absorbent product suitable for the present invention.

FIG. 2 is a cross sectional view through the absorbent article according to the line II-II in FIG. 1.

FIG. 3 a-e illustrates capsules according to an embodiment of the invention having different shapes.

FIG. 4 is a cross section through a capsule according to an embodiment of the invention.

FIG. 5 a-c show a second embodiment of an absorbent article during different stages of exposing the capsules.

FIG. 6 is a schematic view of an apparatus for making the capsules.

### DEFINITIONS

The term “absorbent article” refers to products that are placed against the skin of the wearer to absorb and contain body exudates, like urine, faeces and menstrual fluid. The disclosure mainly refers to disposable absorbent articles, which means articles that are not intended to be laundered or otherwise restored or reused as an absorbent article after use. Examples of disposable absorbent articles include feminine hygiene products such as sanitary napkins, panty liners, tampons and sanitary panties; diapers and pant diapers for infants and incontinent adults; incontinence pads; diaper inserts and the like.

By “probiotic composition” or “bacterial composition” is meant a composition comprising probiotic bacteria, i.e. bacteria that have the ability to reestablish the natural microbial flora of the host. The probiotic composition further comprises a lipid phase.

By “dispersion” is meant a mixture of at least two phases which are insoluble or have a limited solubility in one another and wherein one phase forms solid particles, liquid droplets or gas bubbles in the other phase.

Preferred “lactic acid producing bacteria” include bacteria from the genera Lactobacillus, Lactococcus and Pediococcus. Preferably the selected bacterium used is from the species Lactococcus lactis, Lactobacillus acidophilus, Lactobacillus curvatus, Lactobacillus plantarum or Lactobacillus rhamnosus. More preferably the bacterial strain is selected from Lactobacillus plantarum. Even more preferably the lactic acid producing bacterium is Lactobacillus plantarum 931 (deposition No. (DSMZ): 11918). The bacteria are preferably isolated from the natural flora of a healthy person, preferably the bacteria are isolated from the skin or urogenital area or orally.

By “lipid phase” is meant a water-insoluble organic phase with a fatty character. Lipids suitable to be used in the lipid phase include petroleum-derived lipids, synthetic lipids, and animal- and plant-derived lipids. Preferred lipids are waxes and oils and mixtures thereof.

By “capsule” is meant a structure having a core and a shell.

Examples of “additional components” include, but are not limited to, agents protecting the bacterial cells during drying of the bacteria, agents acting as nutrient for bacterial propagation, and skin caring agents. Further examples of suitable additional components are given below.

### DETAILED DESCRIPTION OF THE PREFERRED EMBODIMENTS

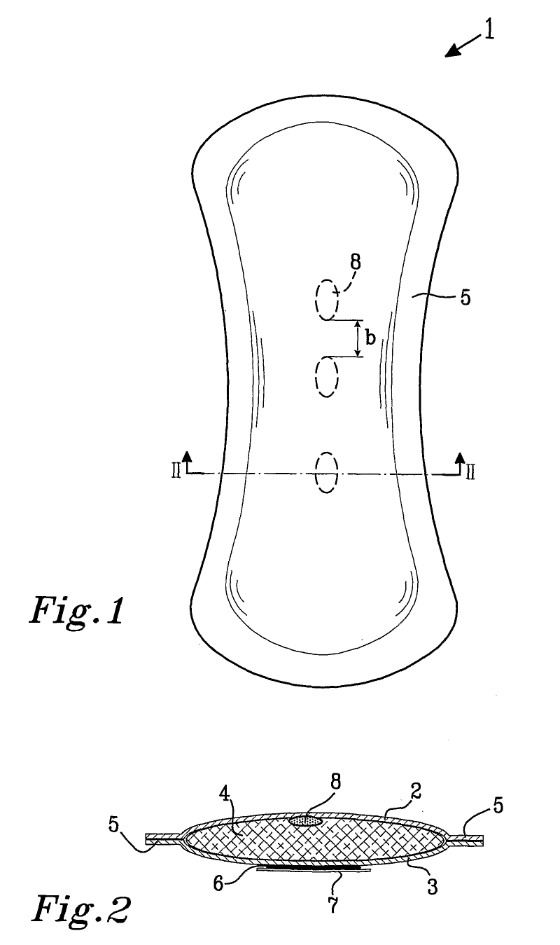

FIGS. 1 and 2 show an embodiment of a sanitary napkin 1 which typically comprises a liquid permeable topsheet 2, a liquid impermeable backsheet 3 and an absorbent core 4 enclosed there between. The liquid permeable topsheet 2 can be composed of a nonwoven material, e.g. spunbonded, meltblown, carded, hydroentangled, wetlaid etc. Suitable nonwoven materials can be composed of natural fibers, such as woodpulp or cotton fibres, manmade fibres, such as polyester, polyethylene, polypropylene, viscose etc. or from a mixture of natural and manmade fibres. The topsheet material may further be composed of tow fibres, which may be bonded to each other in a bonding pattern, as e.g. disclosed in EP-A-1 035 818. Further examples of topsheet materials are porous foams, apertured plastic films etc. The materials suited as topsheet materials should be soft and non-irritating to the skin and be readily penetrated by body fluid, such as urine or menstrual fluid.

The liquid impermeable backsheet 3 may consist of a thin plastic film, e.g. a polyethylene or polypropylene film, a nonwoven material coated with a liquid impervious material, a hydrophobic nonwoven material, which resists liquid penetration or laminates of plastic films and nonwoven materials. The backsheet material may be breathable so as to allow vapour to escape from the absorbent core, while still preventing liquids from passing through the backsheet material.

The topsheet 2 and the backsheet material 3 have a somewhat greater extension in the plane than the absorbent core 4 and extend outside the edges thereof. The layers 2 and 3 are connected to each other within the projecting portions 5 thereof, e.g. by gluing or welding by heat or ultrasonic. The topsheet and/or the backsheet may further be attached to the absorbent core by any method known in the art, such as adhesive or welding by heat or ultrasonic etc. The absorbent core may also be unattached to the topsheet and/or the backsheet.

A fastening means in the form of a region 6 of an adhesive is provided on the side of the backsheet facing away from the wearer in use. The adhesive may releasably attach to the undergarment of the wearer. A release paper 7 protects the adhesive region before use. The adhesive region 6 may have any suitable configuration, such as elongate or transverse strips, dots, full-coated areas etc.

In other embodiments of absorbent articles other types of fasteners, like friction fasteners, tape tabs or mechanical fasteners like hook-and-loop fasteners etc may be used to fasten the articles to the underwear or around the waist of the wearer. Some absorbent articles are in the form of pants and therefore do not need special fastening means. In other cases the absorbent article is worn in special elastic pants without the need for additional fasteners.

The absorbent core 4 can be of any conventional kind. Examples of commonly occurring absorbent materials are cellulosic fluff pulp, tissue layers, highly absorbent polymers (so called superabsorbents), absorbent foam materials, absorbent nonwoven materials or the like. It is common to combine cellulosic fluff pulp with superabsorbents in an absorbent core. It is also common to have absorbent bodies comprising layers of different material with different properties with respect to liquid acquisition capacity, liquid distribution capacity and storage capacity. This is well-known to the person skilled in the art and does therefore not have to be described in detail. The thin absorbent bodies, which are common in today's absorbent articles, often comprise a compressed mixed or layered structure of cellulosic fluff pulp and superabsorbent. The size and absorbent capacity of the absorbent core may be varied to be suited for different uses such as sanitary napkins, pantiliners, adult incontinence pads and diapers, baby diapers, pant diapers, etc.

It is understood that the absorbent article described above and shown in the drawings only represents one non-limiting example and that the present invention is not limited thereto, but can be used in any type of absorbent articles as defined above.

An object of the present invention is to provide hygiene products, such as sanitary napkins, tampons, panty-liners, diapers, incontinence guards, hygiene tissues etc. suitable for absorbing bodily fluids and simultaneously delivering probiotic lactic acid producing bacteria to the skin, or more preferably, the urogenital area. Embodiments of the present invention solve the problems associated with providing products comprising lactic acid producing bacteria, such as problems with bacterial survival, transfer to skin and costs and effectiveness of manufacturing.

The lactic acid bacteria are contained in capsules 8 having certain composition and dimensions to optimize the bacterial survival when applied in an absorbent article. These capsules 8 will be described in greater detail below.

Each capsule 8 comprises a core 9 and a protective shell 10 preventing exposure of the core during transport and storage. The core 9 comprises a dried bacterial composition 11 which is mixed with a lipid phase 12. The hydrophobic character of the lipid phase decreases the amount of air humidity which reaches the bacterial cells dispersed in the lipid phase, thereby increasing the survival time for the bacteria in the bacterial composition. Dispersing the lactic acid producing bacteria in a lipid phase has the additional advantage that transfer of the bacteria to the skin and/or urogenital area is enhanced compared to when no lipid phase is used. When the product is used, the lipid phase softens when exposed to body heat and the bacterial composition is transferred to the skin. When the bacteria come in contact with moisture after delivery to the skin, they are reactivated, start to grow and perform their probiotic action.

Further additives like contact sorption drying carriers, as disclosed in US 2004/0243076, may be present in the bacterial composition. With “contact sorption carriers” are meant substances that have the ability to take up moisture from the ambient environment. Examples of contact sorption drying carriers are, but not limited to, oligo- and polysaccharides and inorganic agents.

Other additives may also be present in the bacterial composition. Examples of such additives include, but are not limited to, agents protecting the bacterial cells during drying of the bacteria, such as sugars (e.g. maltose, glucose, sucrose, trehalose, fructose), proteins (e.g. skim milk, albumin), amino acids (e.g. sodium glutamate), polyols (e.g. xylitol), mannitol and sorbitol, pH-regulating agents (e.g. lactic acid) and antioxidants (e.g. sodium ascorbate). Additional components also include nutrients that enhance bacterial propagation once the bacteria are activated by moisture after they are delivered to the skin or urogenital area. Additional components can also form part of the core. Suitable additional components also include skin caring substances e.g. lipid soluble skin caring substances, such as vitamin A and E, skin caring oils, such as chamomile oils (Bisabolol), eucalyptus oil, lavender oil and phytosterols. The additional components may also comprise a preservation matrix according to WO 98/46261.

The lipid phase 12 of the core 9 is a wax and/or an oil. The wax is preferably chosen from plant waxes, mineral waxes, silicone waxes, animal waxes and mixtures thereof. Preferred mineral waxes are paraffin waxes, micro crystalline waxes, petrolatum and mixtures thereof. One example of a preferred wax is petrolatum. The lipid phase can also be a mixture of wax and oil. Important characteristics of the lipid phase are that it should be possible to disperse the bacterial composition in the lipid phase at a temperature between 20 and 50° C., preferably between 20 and 25° C., and that the water content is low, below 4 weight %, preferably below 2 weight %. The melting behaviour of the lipid phase is a further important factor, which will be discussed in further detail below.

The shell 10 of the capsule 8 should be of a material capable of protecting the core 9 from exposure during transport and storage. It should rupture, melt or be dissolved to expose the core 9 by mechanical actuation force, such as squeezing between fingers, by friction, by body temperature or by being exerted to body liquid or moisture. The shell material may be a wax or a polymer. Examples of suitable shell materials, but not limited thereto, are polyamides, polyurethanes, formaldehyde resins, gelatines, pectins, alginates, waxes and mixtures thereof. The shell material may be water soluble or water insoluble.

The core and the shell may be of the same material, wherein the surface of the core has been hardened, such as by a crosslinking reaction.

The core and shell materials may have different polarities, so that any significant mixture of the materials does not occur in the phase boundary. This is especially important when the core and the shell are made of waxes, i.e. of similar materials.

The capsules 8 are placed in an absorbent article either on the wearer facing side of the topsheet material 2, immediately below the topsheet or close enough below the topsheet to allow the bacterial composition to penetrate through the topsheet to the wearer of the article. An article should contain at least one capsule 8, preferably at least two capsules and more preferably between two and ten capsules placed at certain spaced apart distances from each other. A suitable distance, b, between adjacent capsules 8 would be at least 5 mm. The capsules 8 are preferably spaced apart in the longitudinal direction of the article. Having space apart capsules ensures the contact of probiotic bacteria over a substantial part of the urogenital area. Positioning of the article becomes less sensitive to displacement of the article.

The capsules may alternatively be applied in a transverse direction or in a pattern, such as circles, squares, figurative pattern (e.g. flowers), making the positioning of the article less sensitive.

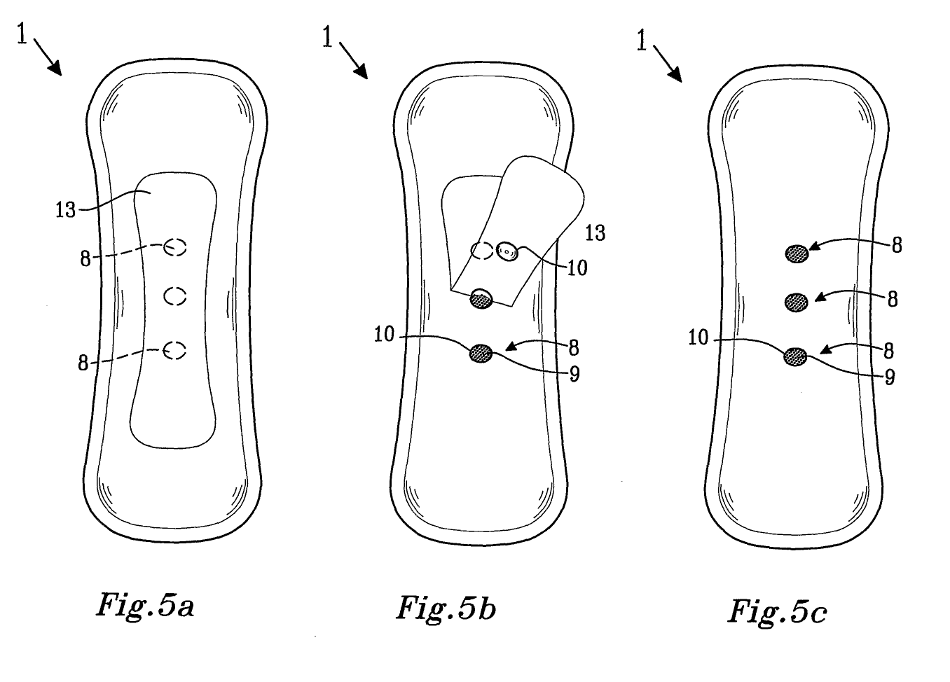

FIG. 5 a-c illustrates an absorbent article in the form of a sanitary napkin, pantiliner, incontinence guard or the like, provided with three capsules, which are spaced apart in the longitudinal direction of the article. The capsules 8 are applied on the wearer facing surface of the topsheet 2 and are before the article is worn (FIG. 5a) covered with a release paper 13, to which the capsules 8 are attached, for example by an adhesive. When the article is to be used the release paper 13 is removed (FIG. 5b), at which the shells 10 of the capsules 8 will burst and the core 9 containing the bacterial composition is exposed (FIG. 5c).

There are of course several alternatives ways of mechanically rupturing the shells of the capsules, for example by integrating a pull string into the capsule and having a free end protruding outside the capsule. By pulling the pull string the capsule will rupture.

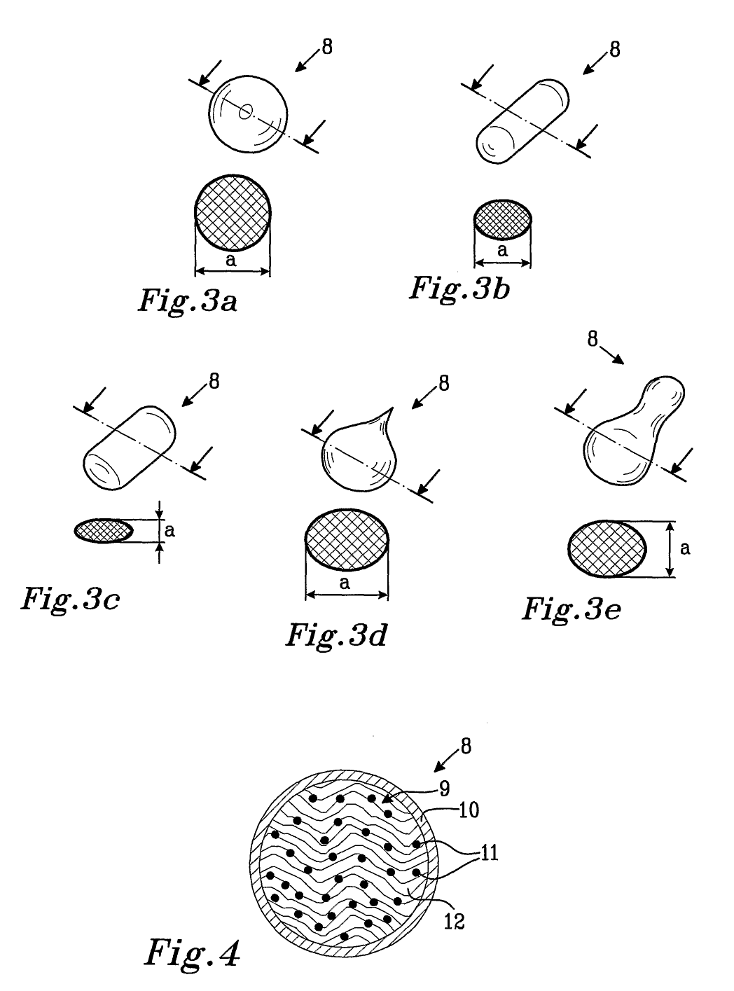

The dimension of the capsule 8 is of importance to ensure a good survival of the bacteria. It is desired that the so called shelf life of article with respect to bacterial survival, should be at least six months. It has been shown that at least a portion of the capsule should have a minimum cross dimension, a, of at least 2 mm, preferably between 4 and 10 mm, more preferably between 4 and 8 mm and most preferably between 5 and 8 mm, in order to ensure a good bacterial survival. The “minimum cross dimension” is herein defined as the cross dimension in the direction in which the capsule has its minimum cross dimension, i.e. is thinnest. This is illustrated in FIG. 3 a-e, wherein FIG. 3a shows a spherical capsule and FIG. 3b a cylindrical capsule, both of which having a substantially circular cross section. In this case the minimum cross dimension, a, is simply the diameter of the capsule. FIG. 3c show a “flat” capsule, wherein the minimum cross dimension, a, is the thickness of the flat capsule. FIG. 3d illustrates a drop-shaped capsule, having a thicker substantially spherical portion with a diameter corresponding to the “minimum cross dimension”, a. FIG. 3e illustrates a capsule having a varying cross dimension over its length, wherein at least one portion of the capsule has a minimum cross dimension or thickness, a, as claimed. Thus there may be other portions of the capsule having smaller cross dimensions than the claimed “minimum cross dimension”.

It has been shown that small minimum cross dimensions of the capsules, in the form of so called micro capsules, result in a poor bacterial survival. It was especially shown that after 3-6 months the bacterial survival of Lactobacillus dispersed in petrolatum in aluminium wells of a depth of 1 mm was significantly lower than for those dispersed in petrolatum in aluminium wells having a depth of 3 and 5 mm. In all cases the diameter of the wells were 17 mm. This test is illustrated in Table 1 below.

TABLE 1

| Depth of | Amount | Amount of | | | | | | |
|---|---|---|---|---|---|---|---|---|
| wells | petrolatum | Lb 0-value | | | | | | |
| (mm) | (mg) | (Cfu/g) | 1 month | 2 mon. | 3 mon. | 4 mon. | 5 mon. | 6 mon. |
| 1 | 200 | 1.5E8 | 7.5E7 | 1.5E7 | 2.9E6 | 4.2E5 | 1.7E4 | 6.6E3 |
| 3 | 600 | 1.5E8 | 6.3E7 | 3.0E7 | 4.4E7 | 2.0E7 | 2.0E7 | 1.8E7 |
| 5 | 1000 | 1.5E8 | 8.0E7 | 5.0E7 | 5.8E7 | 4.6E7 | 2.1E7 | 3.4E7 |

### Preparation of the Capsules

Preferred ways of preparing the capsules will be described below. A water suspension of at least one lactic acid producing bacterial strain having a concentration of 10⁶-10¹⁵ CFU (colony forming units)/ml, preferably 10¹⁰-10¹³ CFU/ml is prepared. The suspension may also contain additional components like contact sorption drying carriers, nutrients and/or protecting agents. Examples of such additional components are given above.

Lactic acid producing bacteria are chosen due to their positive effect in preventing and treating microbial infection in the urogenital area and on the skin. The bacteria are preferably isolated from a healthy person, preferably from the skin or urogenital area of a healthy person. Preferred “lactic acid producing bacteria” include bacteria from the genera Lactobacillus, Lactococcus and Pediococcus. Preferably the selected bacteria are from the species Lactococcus lactis, Lactobacillus acidophilus, Lactobacillus curvatus or Lactobacillus plantarum. More preferably the selected bacterium is a Lactobacillus plantarum strain. Even more preferably the lactic acid producing bacterium is Lactobacillus plantarum 931 (deposition No. (DSMZ): 11918). The lactic acid producing bacteria can be provided alone or in mixtures containing at least two bacterial strains.

The suspension is dried using any of the following techniques: convective drying methods, contact drying methods or by using electromagnetic radiation or freeze drying. Examples of suitable convective drying methods include spray drying, spray granulation and fluidized bed drying. The common feature for convective drying methods is that warm and dry gas flushes around the product and enters into a heat and mass transfer with the product. Convective methods transfer required heat and/or dryness by convection to the wet product. During contact drying, the wet product is stationary in touch with a warm surface or constantly brought into new contact with the warm surface by stirring or revolving. Drying by electromagnetic radiation (infrared or microwave radiation) involves using a belt dryer or a stationary support and submitting the wet product to electro-magnetic radiation energy which is being absorbed by the wet product. The absorbed energy serves to warm up the product whereby the moisture in the wet product is evaporated. Drying times using electromagnetic radiation often result in very short drying times. After drying the powder may be used directly or be refined, e.g. grinded.

A probiotic composition is thereafter prepared wherein the dry bacterial composition, comprising lactic acid producing bacteria and optional additional components, is dispersed in a lipid phase which will form the core 9 of the capsules 8. During this step further additional components, such as nutrients for bacterial propagation and skin caring substances can be added to the probiotic composition.

The lipid phase can be composed of a single lipid or a mixture of two or more lipids. The lipid phase due to its hydrophobic character works as a water vapor barrier, thus makes sure that a very low water activity is maintained during storage. In table 2 below a selection of lipids suitable for the core 9 of the capsules 8 is presented.

TABLE 2

| Name | Producer | Melting range | Main ingredients |
|---|---|---|---|
| Caremelt 107 | Cognis¹⁾ | 25-58° C. | Triglyceride, paraffin, |
| | | | monoglyceride |
| Caremelt 3 | Cognis | 30-47° C. | Triglyceride, paraffin, |
| | | | silicone wax, liquid |
| | | | triglyceride |
| Caremelt 58 | Cognis | 30-49° C. | Triglycerides, |
| | | | polymer wax, |
| | | | stearyl alcohol, |
| | | | silicone wax |
| Vaseline | AC Hud AB²⁾ | 5-45° C. | Petrolatum |
| Beeswax | Apoteket³⁾ | | Cera Flava |
| Akosoft 36 | Karlshamn⁴⁾ | 34-38° C. | Vegetable fat, hard fat |
| Lipex BC | Karlshamn | 35° C. | Hydrogenated |
| | | | Vegetable oil |
| AMS-C30 | DOW-corning⁵⁾ | 70° C. | Silicone wax |

¹⁾Henkel KgaA, Dusseldorf, Germany
²⁾Aco Hud AB, Stockholm, Sweden
³⁾Apoteket AB, Produktion och Laboratorier, Gothenburg, Sweden
⁴⁾Karlshamns AB, Karlshamn, Sweden
⁵⁾Seneffc, Belgium

The probiotic composition is used in a hygiene absorbent product. For this purpose it is important that the lipid phase has a melting behavior that allows the lipid phase to support bacterial survival and not disturb the absorptive power of the hygiene product.

There is also an upper temperature limit for the melting behavior of the lipid phase. This limit is in part governed by the fact that for mixing the bacterial composition with the lipid phase, the lipid phase has to be soft enough in order to obtain a homogenous mixture. A lipid phase with a too high melting point has to be brought to temperatures that are too high for the bacteria to withstand while being mixed with the lipid phase and therefore a too large portion of the bacteria would die during preparation of the probiotic composition. Also, a lipid phase with too high melting point is not suitable since it does not soften to a high enough extent when in contact with the skin and therefore delivery of the bacteria to the skin is impaired.

The consistency of the core material is influenced by the ratio of bacterial composition to the lipid phase which provide texture and consistency of the core material. By varying the ratio of amount of bacterial composition to amount of lipid phase it is possible to achieve a core material with a suitable consistency.

Therefore, preferably, the lipid phase is in major part solid at room temperature and up to 30° C., becomes softer at body temperature between 30° C. and 40° C., and melting above 60-70° C. However, the lipid phase is still exhibiting soft properties from 30° C. This melting behavior can be achieved by using a single lipid or by mixing different lipids with different melting behaviors in order to achieve the desired melting behavior of the lipid phase.

In order for the lipid phase not to interfere with bacterial survival the water content should be low, below 4% by weight, preferably below 2% and more preferably below 1% by weight. The water vapor transmission rate of the lipid phase, measured according to ASTME 398-83 at 37.8° C. (100° F.) and 90% relative humidity (RH), is 10 g/m²/24 h or less, more preferably 5 g/m²/24 h or less, most preferably 2 g/m²/24 h or less.

The shell 10 of the capsules should be of a material capable of protecting the core 9 from exposure during transport and storage. It should rupture, melt or be dissolved to expose the core 9 by mechanical actuation force, such as squeezing between fingers or by friction, by body temperature or by being exerted to body liquid or moisture. The shell material may be a wax or a polymer. Examples of suitable shell materials, but not limited thereto, are polyamide, polyurethane, formaldehyde resin, gelatine, pectin, alginate, wax and mixtures thereof. The shell material may be water soluble or water insoluble.

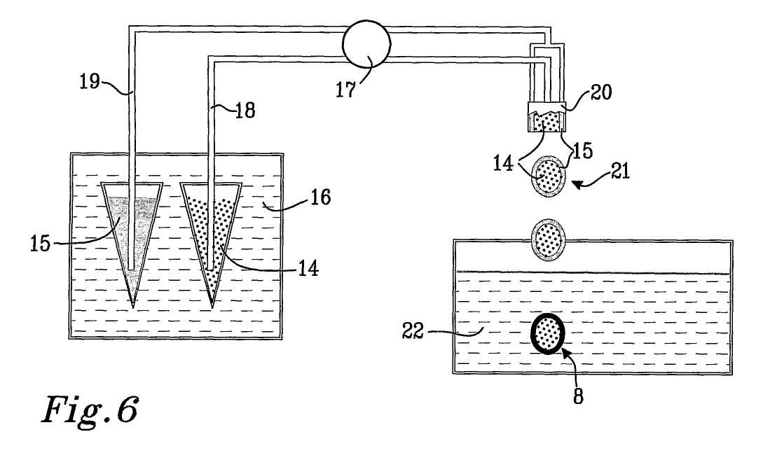

Encapsulation of the core 9 by the shell 10 can be accomplished by different techniques. One example of a suitable technique is the double nozzle technique, in which the core material and the shell material are pumped in liquid form through a concentric double nozzle, which produces droplets. The droplets are composed of an inner phase, the core, and an outer phase, shell material. The outer phase may be solidified by forming crosslinks and/or by subsequent drying and/or by decrease of temperature. An example of an apparatus for preparing the capsules is illustrated in FIG. 6. The core material, i.e. a mixture of the lipid phase and the bacterial composition and optional additives, and a coating material for forming the shell, are placed in two different vessels 14 and 15 in a water bath 16 at a suitable temperature, e.g. 40° C., at a sufficient time period for the core and the shell materials to form a pumpable liquid phase. The two liquid phases are then pumped, by means of a pump 17, through a pair of conduits 18 and 19, to a double nozzle 20. The conduits 18 and 19 may be thermostated to maintain a desired temperature of the liquids pumped there through. Drops are formed by the nozzle 20, having dimensions larger than the nozzle. The dimensions of the double nozzle should be adapted to produce drops 21 having the desired dimensions. The drops have an inner core of the lipid phase with dispersed bacterial composition and optional additives and a shell of coating material. The drops may fall into a bath 22 containing cooling liquid, with optional additives like cross-linking initiators, to accomplish cross-linking of the shell. The thus formed capsules 8 may undergo additional treatment, like drying, coating with additional materials, like gelatine, polyethylene glycol etc.

In an alternative embodiment the bath 22 is excluded.

### EXAMPLE

A probiotic powder containing Lactobacillus plantarum 931 was prepared having an average concentration of 5·10¹¹ CFU/g. The probiotic powder was mixed with petrolatum wax by slow addition of the powder into the petrolatum previously warmed up to 30° C. The final concentration of LB931 was 10% by weight and 90% by weight petrolatum. Different coating materials were tested: alginate, a mixture of alginate and gelatin, and a mixture of pectin and alginate.

Capsules were produced using the double nozzle technique as illustrated in FIG. 6. The concentric nozzle had the following dimensions: inner diameter: 700 μm and outer diameter: 1500 μm. The size of the drops formed were between 4.5 and 5.5 mm. The bath 22 contained a CaCl₂-solution at room temperature in which the coating material was instantaneously cross-linked. Drying of the thus formed capsule was performed by air drying or tumbler drying at 35° C. The capsules shrunk slightly after drying. Once the capsules were dried, their content could be released by squeezing them between the fingers. Talc may optionally be added to the capsules to make them less sticky. A bacterial count test was performed after 48 hours. This test showed a very high bacterial viability (>50%) for the capsules having an alginate shell and the capsules having a gelatin/alginate shell. Capsules prepared with a pectin/alginate shell showed a viability of about 30%.

Thus, while there have been shown and described and pointed out fundamental novel features of the invention as applied to preferred embodiments thereof, it will be under-stood that various omissions and substitutions and changes in the form and details of the devices, method steps and products illustrated may be made by those skilled in the art. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. An absorbent article comprising: at least one capsule, at least one portion of which has a minimum cross dimension of at least 2 mm, said capsule containing a bacterial composition in a lipid phase.

2. The absorbent article as claimed in claim 1, wherein said bacterial composition comprises at least one lactic acid producing bacterial strain.

3. The absorbent article as claimed in claim 1, wherein said capsule has at least one portion with a minimum cross dimension of between 2 and 10 mm.

4. The absorbent article as claimed in claim 1, wherein said article is provided with at least two capsules.

5. The absorbent article as claimed in claim 3, wherein said article is provided with no more than ten capsules.

6. The absorbent article as claimed in claim 4, wherein said capsules are located spaced apart a distance of at least 5 mm in the article.

7. The absorbent article as claimed in claim 6, wherein said capsules are spaced apart in the longitudinal direction of the article.

8. The absorbent article as claimed in claim 1, wherein said capsule comprises a core containing said bacterial composition in said lipid phase and a shell preventing exposure of the core during transport and storage, wherein the core and shell are of different materials.

9. The absorbent article as claimed in claim 1, wherein said lipid phase is a wax and/or an oil.

10. The absorbent article as claimed in claim 9, wherein the wax is chosen from a plant wax, a mineral wax, an animal wax, a silicon wax and mixtures thereof.

11. The absorbent article as claimed in claim 10, wherein the wax is a mineral wax.

12. The absorbent article as claimed in claim 11, wherein the mineral wax contains petrolatum as a main component.

13. The absorbent article as claimed in claim 8, wherein the shell is of a material that ruptures, melts or is dissolved by mechanical actuation force or friction, by body temperature or by being exerted to body liquid or moisture.

14. The absorbent article as claimed in claim 13, wherein the shell material is a wax or a polymer.

15. The absorbent article as claimed in claim 14, wherein the shell material is chosen from the following materials: polyamide, polyurethane, formaldehyde resin, gelatine, pectin, alginate, plant wax, mineral wax, animal wax, silicon wax and mixtures thereof.

16. The absorbent article as claimed claim 8 wherein the core material and the shell material have different polarities, so as to prevent significant mixture of said materials in the phase boundary between the shell and the core.

17. The absorbent article as claimed in claim 1, wherein said at least one capsule is applied on the wearer facing side of the article or below one or more layers of the article permitting penetration of the lactic acid producing bacterial strain to the wearer.

18. The absorbent article as claimed in claim 17, wherein said at least one capsule is applied on the wearer facing side of the article and is covered by a releasable material layer, to which it is at least partly adhered, so that upon removal of the releasable material layer the shell of the capsule will burst and expose the core material.

19. A method of making a capsule containing a bacterial composition in a lipid phase, said capsule having a core and a shell, the method comprising: delivering the materials which form the core and the shell through a concentric double nozzle, which produces droplets composed of an inner phase containing the core material and an outer phase containing the shell material, said droplets forming the capsules.
